(19) United States
(12) Patent Application Publication
(10) Pub. No.: US 2002/0143166 A1
(43) Pub. Date: Oct. 3, 2002

(54) Method for removing a universal linker from an oligonucleotide

(75) Inventors: Richard M. Pires, Damascus, MD (US); Gulilat Gebeyehu, Frederick, MD (US)

Correspondence Address: STERNE, KESSLER, GOLDSTEIN & FOX PLLC, 1100 NEW YORK AVENUE, N.W., SUITE 600, WASHINGTON, DC, 20005-3934, US

(73) Assignee: Invitrogen Corporation

(21) Appl. No.: 09/986,750

(22) Filed: Nov. 9, 2001

Related U.S. Application Data
(60) Provisional application No. 60/246,713, filed on Nov. 9, 2000.

(51) Int. Cl. C07H 21/04

(52) U.S. Cl. 536/23.1; 536/25.3

(57) ABSTRACT

The invention relates to a method for cleavage of a linker from an oligonucleotide comprising contacting an oligonucleotide-linker conjugate with a gaseous nucleophilic cleavage reagent under conditions that result in the cleavage of the linker from the oligonucleotide.

### BACKGROUND OF THE INVENTION

1. Field of the Invention

The present invention relates to processes for the substantial cleavage of a linker from an oligonucleotide comprising contacting an oligonucleotide-linker conjugate with a gaseous nucleophilic reagent such as ammonia.

2. Related Art

A variety of solid phase oligonucleotide synthesis techniques are known to those skilled in the art. Such techniques include phosphoramidite, phosphotriester, phosphodiester, phosphite and H-phosphonate methods and the like, each of which is generally known in the fields of chemistry, biochemistry and molecular biology. For example, the β-cyanoethyl phosphoramidite method is described in U.S. Pat. No. 4,458,066 issued to Caruthers, et al., entitled “Process for Preparing Polynucleotides,” which is incorporated herein by reference.

Currently, most standard procedures used in the chemical synthesis of DNA rely upon controlled pore glass (CPG) that is pre-functionalized with the base corresponding to the 3′-end of the oligonucleotide to by synthesized. This requires the use of four different CPG's, with the specific CPG used depending on the desired base at the 3′-terminus of the oligo being synthesized (FIG. 1). On a standard DNA synthesizer this causes little inconvenience; however, this standard scheme is much more problematic when used in conjunction with high throughput DNA synthesis instruments which utilize 96 well plates to generate many different oligos simultaneously. The difficulty of loading the correct CPG in each of the 96 wells is coupled with the danger of incorrectly loading one or more of the wells with the wrong CPG. In addition, having a different support for each base increases the number of raw materials that must be stocked and managed. Thus, the development of a system where a single CPG is compatible with any base at the 3′-end of the oligo, is highly desirable.

A number of linkers, termed universal linkers, have been developed to couple the 3′-terminal base with a solid support, e.g. CPG, allowing a single CPG to be used in the synthesis of olignucleotides with any base at the 3′-end (FIG. 2). Most of the commercially available linkers contain a cyclic vicinal diol, to which the first base is coupled. Upon cleavage and deprotection, the oligo is cleaved from the linker, and the 3′-phosphate is removed by the formation of a cyclic phosphodiester (FIG. 3). Generally this cleavage and deprotection requires heating the oligo for an extended period of time (˜18 hours) with concentrated aqueous ammonia, or the use of concentrated NH₄₀H in conjunction with a salt additive, such as LiCl which requires an additional step for removal. In addition, the cleavage and deprotection can be accomplished with ammonium hydroxide /methylamine (AMA), but this reagent requires the use of a special protecting group on dC to avoid incorporation of methylamine into the loligo.

It is clear that the use of a universal linker, while desirable, is impractical due to the drastic conditions and the length of time currently required to cleave the oligo from the universal linker. When using a universal linker in oligonucleotide synthesis, there are at least three reactions which occur simultaneously during the cleavage and deprotection step. First the ester bond between the universal linker and the solid support is cleaved. Second, the exocyclic amino groups on the oligonucleotide are deprotected. And finally, the phosphodiester bond between the universal linker and the 3′-terminal base of the newly synthesized oligonucleotide is cleaved (FIG. 4). The first two of these reactions occur relatively rapidly (˜1 hr); however, the cleavage of the universal linker from the oligonucleotide is a slowprocess, usually necessitating an 18 hour incubation with the liquid cleavage and deprotection reagent. Additionally, there generally has to be an accompanying step to remove the free universal linker product. Because of these problems few oligonucleotide manufacturers use universal linkers, despite the obvious advantages.

Biosearch Technologies, Inc. has recently introduced a new generation of vicinal diol containing universal linkers which remain bound to the solid support during deprotection and cleavage (Lyttle et. al., Nucleosides and Nucleotides 18: 1809-1824 (1999); FIG. 5). While this addresses the issue associated with removal of the contaminating linker from the final oligonucleotide solution, this new generation of universal linker still requires an extended treatment with hot ammonium hydroxide to obtain full cleavage and deprotection.

U.S. Pat. No. 5,514,789 describes a method for the cleavage and deprotection of newly synthesized oligonucleotides from standard solid supports using a gaseous cleavage/deprotection reagent such as gaseous ammonia, ammonium hydroxide vapors, or methylamine.

It has now been discovered that the use of gaseous nucleophilic amino compounds is a rapid and effective way to cleave newly synthesized oligonucleotides from the linkers attaching them to a solid substrate. This new method reduces the time needed for cleavage/deprotection from approximately 18 hours to less than 2 hours, making the use of universal linkers in high throughput oligonucleotide synthesis more efficient.

### SUMMARY OF THE INVENTION

The invention relates to a method for substantially cleaving a linker, which attaches an oligonucleotide to a solid phase, from an oligonucleotide to give free oligonucleotide comprising contacting an oligonucleotide-linker-solid phase conjugate with an effective amount of a gaseous nucleophilic amino compound under conditions that result in the removal of the linker, thereby yielding the free oligonucleotide.

Specifically, the invention relates to a method for cleavage of a linker from an oligonucleotide, comprising contacting a conjugate comprising an oligonucleotide; a vicinal diol containing linker, which is not the 3′-terminal nucleotide; and a solid support with a gaseous nucleophilic composition under conditions that result in the cleavage of an ester linkage between the first constituent of the oligonucleotide (usually the 3′-OH of the 3′ terminal nucleotide) and the phosphate of the linker, resulting in the cleavage of the oligonucleotide from the linker. Upon removal of the linker from the oligonucleotide, the linker forms phosphorous containing heterocycle, most preferably a cyclic phosphodiester. More specifically, the invention relates to the cleavage of one or more oligonucleotides (of the same or different sequences), being liberated from one or more universal linkers (having the same or different structures) using one or more gaseous nucleophillic amino compounds (having the same or different structures). In comparison to methods for cleaving an oligonucleotide from a solid support, the method of the present invention relates to cleavage of an oligonucleotide from a linker, particularly a universal linker; whereas methods for cleavage from a solid support involve the cleavage of oligonucleotides which are directly bound to the solid support (FIGS. 1A and 1B).

In a preferred embodiment, the reaction of the oligonucleotide, linker, solid support conjugate with the cleavage reagent takes place at a temperature between about room temperature and about 150° C., for between about 1 and about 240 minutes.

In a most preferred embodiment, the oligonucleotide, linker, solid support conjugate will be reacted with hydrated ammonia vapors at about 95 ° C. for about 120 minutes. The cleaved oligonucleotide is then isolated by washing the solid phase with water or aqueous buffer.

The oligonucleotide, linker, solid support conjugate may have the following general structure:

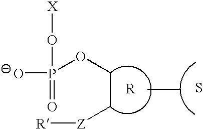

wherein X is the termini of the oligonucleotide (usually the 3′ nucleotide), S is a solid support, R is an optionally substituted tetrahydrofiran, phenyl or cyclopentane ring, and R′ is a protecting group and Z is O S or Se. Examples of linkers are shown in FIG. 2. Substitutions on the R group, if present, may include hydroxyls, amino, thiols, esters, amides, nitrogenous bases and other functional groups.

### BRIEF DESCRIPTION OF THE DRAWINGS

FIGS. 1A and 1B depict schemes showing the general methods of oligonucleotide synthesis using the standard methodology and universal linker methodology.

FIG. 2 shows examples of the structures of commercially available universal linkers.

FIG. 3 depicts the mechanism of cleavage of an oligonucleotide from a universal support showing the cyclic phosphodiester and free nucleotide products.

FIG. 4 is a scheme illustrating the problems associated with the use of universal linkers in high throughput automated synthesis. The process labeled “Removal of Universal Linker” takes approximately 18 hours.

FIG. 5 depicts a scheme showing the reaction mechanism of the second generation of universal linkers. These linkers are described in Lyttle et. al. Nucleosides and Nucleotides. 18: 1809-1824 (1999).

FIGS. 6A and 6B depict HPLC chromatograms comparing the cleavage and deprotection using concentrated ammonium hydroxide at 95° C. and 75 min (FIG. 6A) with gas phase cleavage and deprotection of a 20-mer at 95° C., 80 psi, and 60 min (FIG. 6B).

FIG. 7 depicts a mass spectrograph of an oligonucleotide cleaved and deprotected using a gas phase process for 2 hours.

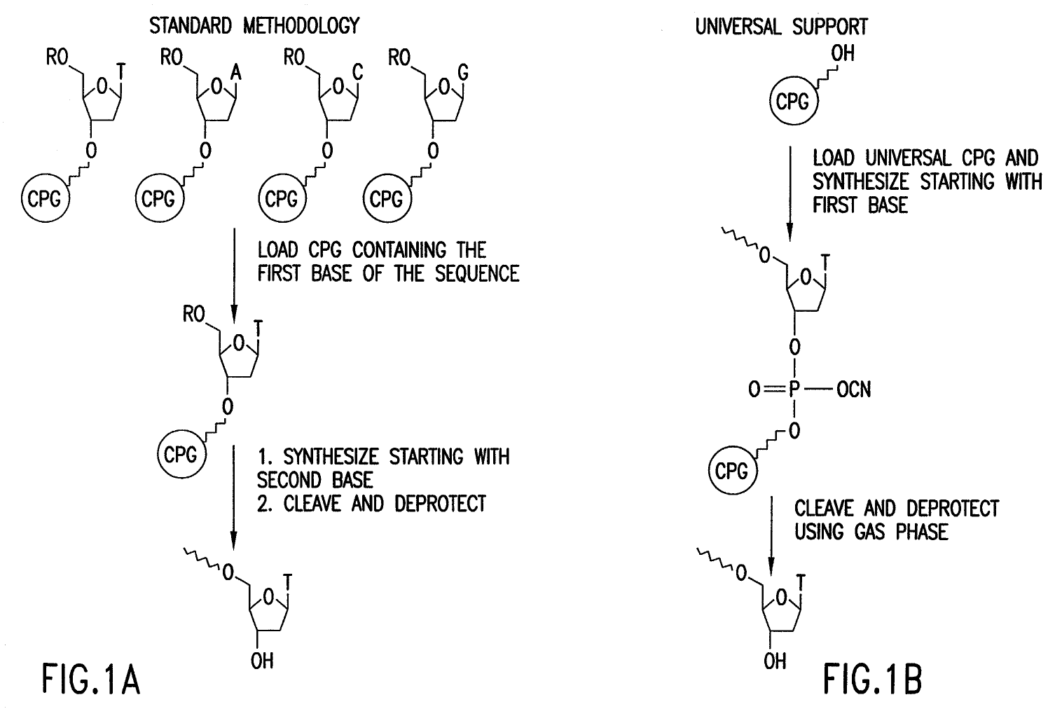

### DETAILED DESCRIPTION OF THE INVENTION

Definitions

In the description that follows, a number of terms used in the fields of chemistry, biochemistry and molecular biology are utilized extensively. In order to provide a clearer and consistent understanding of the specification and claims, including the scope to be given such terms, the following definitions are provided.

Universal linker. As used herein, the term refers to a molecule which functions in attaching a nucleotide or oligonucleotide to a solid phase support, wherein that linker molecule is not the 3′-terminal nucleotide of the oligonucleotide being synthesized. Distinguishing features of universal supports include, but are not limited to the ability to attach the desired 3′-terminal nucleotide directly to the universal linker, which may then be attached to the solid phase. Usually this linkage comprises a phosphodiester linkage to the 3′-hydroxyl of the 3′-terminal nucleotide. Upon completion of oligonucleotide synthesis and removal of the universal linker with a nucleophilic reagent, the 3′-hydroxyl of the terminal nucleotide is regenerated and the phosphate is bound to the universal linker forming a cyclic phosphodiester.

Cleavage or removal of the linker. As used herein, the phrase refers to the substantial cleavage of the ester linkage between the terminal component of the oligonucleotide, preferably the 3′-hydroxyl of the terminal nucleotide and the phosphate moiety forming a free oligonucleotide comprising an intact 3′-hydroxyl group, and a linker comprising a phosphorous containing heterocycle, most preferably a cyclic phosphodiester. Cleavage is considered to be substantial if at least 80%, and preferably 90% or greater, of the isolated oligonucleotides do not contain an attached linker, as measured for example by HPLC, after contact with the cleavage reagent. This does not require cleavage of the linker from the solid support, which is a separate reaction occurring simultaneously.

### Description of Preferred Embodiments

The invention relates to a method for substantially cleaving a linker which attaches an oligonucleotide to a solid phase support from an oligonucleotide comprising contacting a linker-oligonucleotide-solid phase conjugate with an effective amount of a gaseous cleavage reagent such as a gaseous, nucleophilic amino compound.

Specifically, the invention relates to a method for cleavage of a linker from an oligonucleotide, comprising contacting a conjugate comprising an oligonucleotide; a vicinal heteroatom (e.g., a vicinal diol, vicinal amino alcohol, or a vicinal thiol alcohol) containing linker, which is not the 3′-terminal nucleotide; and a solid support with a gaseous nucleophilic composition under conditions that result in the cleavage of an ester linkage between the 3′-OH of the oligonucleotide and the phosphate of the linker, resulting in the cleavage of the oligonucleotide from the linker. Upon removal of the linker from the oligonucleotide, a phosphorous containing heterocycle is produced, most preferably a cyclic phosphodiester. In a most preferred aspect of the invention, the linker is a universal linker.

In a preferred aspect of this embodiment, on the oxygen of the vicinal diol not bound to the phosphate of the 3′-terminal nucleotide of the oligonucleotide, is bound a protecting group. Such acceptable protecting groups include DMTr, acyl, aryl, silyl, trifluoroacetyl, benzyl, or substituted benzyl or aryl groups.

In a preferred embodiment, the reaction of the oligonucleotide, linker, solid support conjugate with the cleavage reagent takes place at a temperature between about room temperature and about 150° C., for between about 1 minute and about 5 hours.

In a most preferred embodiment, the oligonucleotide, linker, solid support conjugate is reacted with ammonia vapors at about 95° C. for about 120 minutes. The free oligonucleotide is isolated by washing the solid phase with water or aqueous buffer.

The present invention provides significant improvement over existing methods for removal of linkers, particularly universal linkers, from oligonucleotides. Specifically, the invention allows for the removal of universal linkers from oligonucleotides in 0-5 hours, most preferably 1-2 hours, as opposed to the existing methods which require at least 18 hours for substantially complete cleavage; moreover, this decrease in linker removal time makes the use of universal linkers in high throughput oligonucleotide synthesis more efficient.

As discussed above, cleavage from the solid support, deprotection, and removal of a universal linker are normally accomplished in the same reaction with a liquid cleavage/deprotection reagent, such as liquid ammonium hydroxide. The major problem with this method is the length of time needed to remove the linker from the oligonucleotide (˜18 hours). This fact has kept universal linkers from being used widely in high throughput oligonucleotide synthesis, despite the advantage of only having to use a single solid support if a universal linker is used. It has been discovered that by using a gas phage cleavage reagent, the time needed to cleave the linker from the oligonucleotide is reduced to 0-5 hours, most preferably 1-2 hours.

The nucleophilic amino compound may be ammonia vapors (e.g. obtained by heating a sealable chamber having a quantity of ammonium hydroxide in the bottom), or a C₁₋₆ alkylamino compound. The alkyl group may be straight or branched chain. Examples of such alkylamino compounds include methylamine, ethylamine, propylamine, isopropylamine, butylamine, sec-butylamine, pentylamine and hexylamine. Alternatively, the nucleophillic amino compound could be any number of compounds containing a nucleophillic moiety capable of reacting in the gas phase (e.g., sodium methoxide, hydrogen sulfide, certain hydroxides, or alkoxides). The oligonucleotide is not soluble in the nucleophillic amino compound and, thus, the nucleophillic amino compound may be removed by filtration. The DNA, which remains bound to the solid support during filtration, can then be eluted with an aqueous buffer.

The oligonucleotides may be prepared by well known methods, e.g. the phosphoramidite, phosphotriester, phosphodiester, phosphite and H-phosphonate methods, each of which are generally known in the field of chemistry, biochemistry and molecular biology. For example, the β-cyanoethyl phosphoramidite method is described in U.S. Pat. No. 4,458,066 issued to Caruthers, et al., entitled “Process for Preparing Polynucleotides,” which is incorporated herein by reference. See also E. Eckstein (ed.), Oligonucleotides and Analogs, A Practical Approach, IRL Press, Oxford (1991); GB 2,125,789;

and U.S. Pat. Nos. 4,415,732, 4,739,044 and 4,757,141. Such oligonucleotides may be DNA, RNA, mixture of DNA and RNA, derivatives of DNA and RNA, and mixtures thereof.

In the most preferred embodiment of the invention the oligonucleotide is attached to the universal linker by a phosphodiester linkage to the 3′-hydroxyl of the 3′-terminal nucleotide. The linker may also be attached to the solid phase support, typically by an ester linkage. If the linker is removed from the oligonucleotide while the linker is attached to the solid support, the ester linkage between the solid support and the linker will also be cleaved by the gaseous cleavage agent. The removal of the linker will cause the release of the oligonucleotide which may then be recovered by washing the solid phase with water or a buffer.

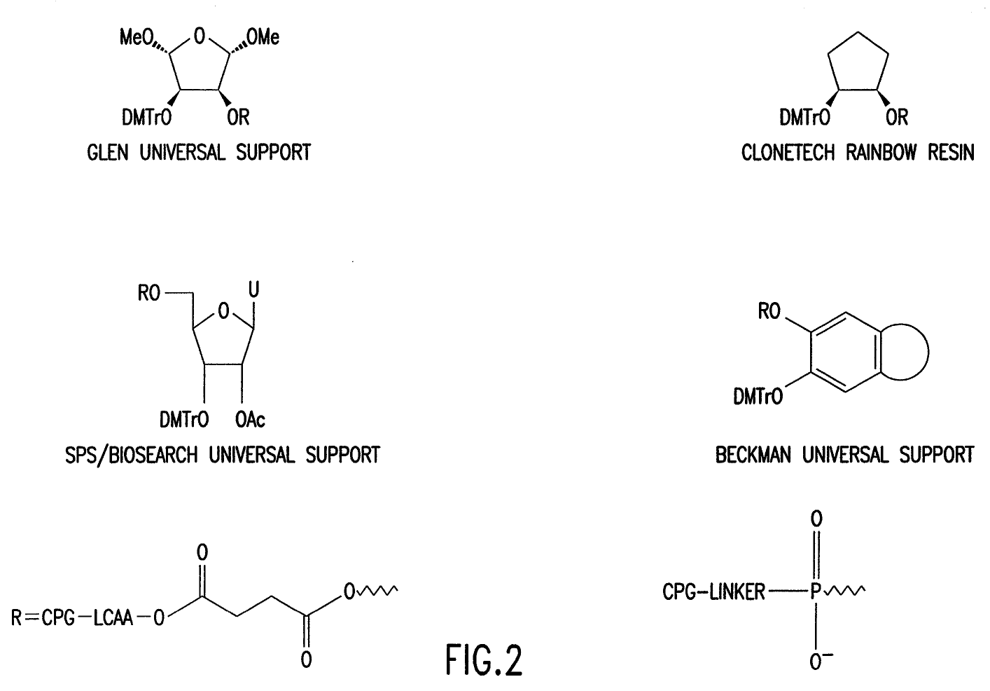

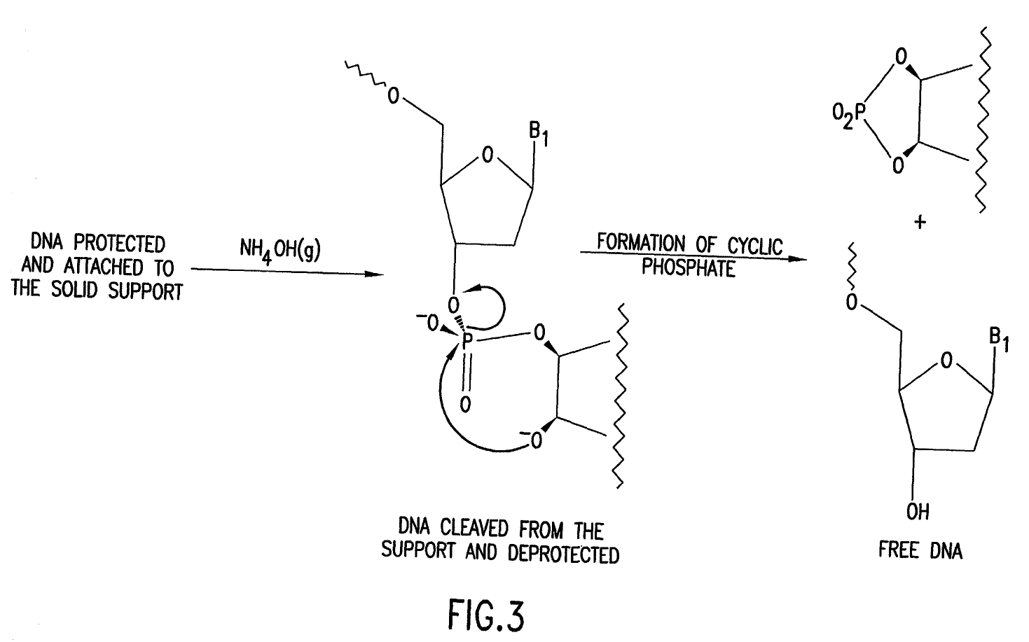

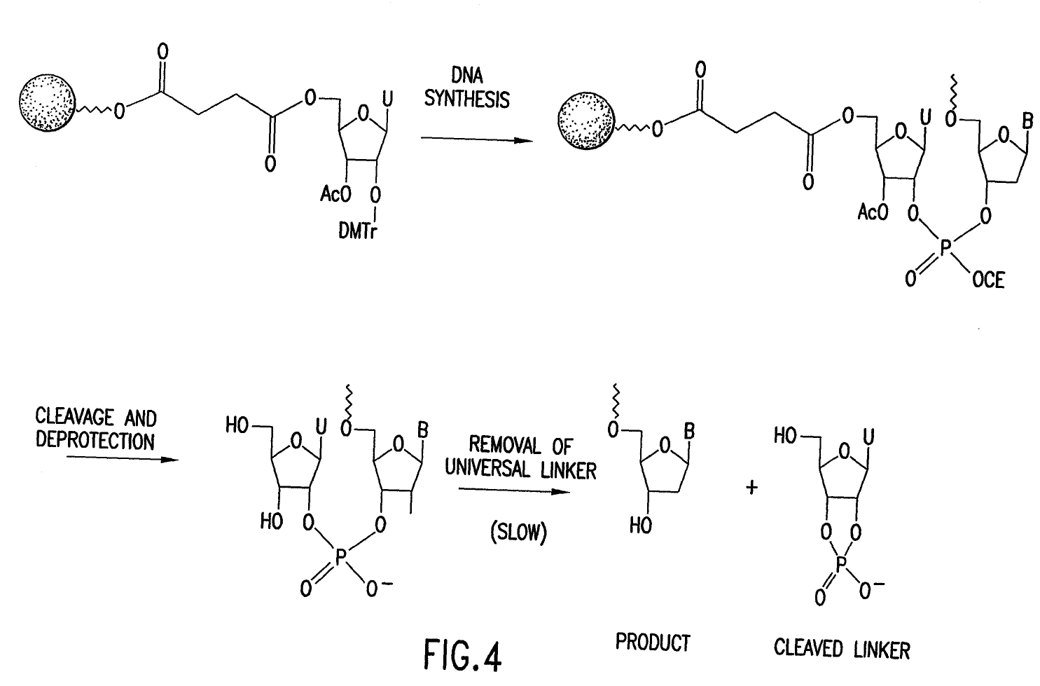

Universal linkers have been described in a number of publications (Nelson et. al. Biotechniques. 22: 753-756 (1997); Gough et. al. Tetrahedron Let. 24: 5321-5324 (1983); and Lyttle et. al. Nucleosides Nucleotides. 18: 1809-1824 (1999)). The advantage of universal linkers over traditional methods for oligonucleotide synthesis is the ability to add the desired 3′-terminal nucleotide of the oligonucleotide by automated coupling of the corresponding phosphoramidite directly to the linker, as opposed to using four different supports, each corresponding to a desired 3′-terminal base. When synthesis of the oligonucleotide is complete, the 3′-hydroxyl is regenerated and the 3′-phosphate remains attached to the universal linker in the form of a cyclic phosphodiester. A number of universal linkers are commercially available, but their use has been limited due to the need for prolonged incubation times to remove then from the oligonucleotide after synthesis is complete. Many of these linkers are sold pre-attached to the solid matrix, some examples include, but are not limited to products from Glen, Clontech, SPS/Biosearch, and Beckman (FIG. 2).

The removal of the linker is preferably carried out in a sealable chamber (although an open chamber may be used in accordance with the invention) that can be heated. Such sealable chambers include screw cap vials, Parr bottles, and the like. The oligonucleotide synthesis and cleavage from the support may be carried out with a commercially available DNA synthesizer, e.g. the ABI 380B DNA synthesizer, or other equipment that is set up for high throughput synthesis on a multi well channel, e.g. a 96 well plate (see, e.g., U.S. application No. 245,023, filed Feb. 5, 1999, which is incorporated herein by reference in its entirety).

The gaseous nucleophilic cleavage reagent is present in an amount effective to cleave the linker from the oligonucleotide. In general, the gaseous nucleophilic cleavage reagent is present in a large excess compared to the oligonucleotide. In accordance with the invention, any number of nucleophillic amino compounds can be used. In the case of ammonia, the sealable chamber may be charged with about 20 to 200 psi of ammonia, most preferably, about 80 psi. Optimal amounts of the liquid alkylamino compounds, from which the gas is commonly generated, may be determined with no more than routine experimentation; likewise, the gas phase of the nucleophillic amino compound can be used directly.

The following examples are illustrative, but not limiting, of the method and compositions of the present invention. Other suitable modifications and adaptations of the variety of conditions and parameters normally encountered in molecular biology and chemistry, particularly oligonucleotide synthesis, which are obvious to those skilled in the art in view of the present disclosure are within the spirit and scope of the invention.

### Example

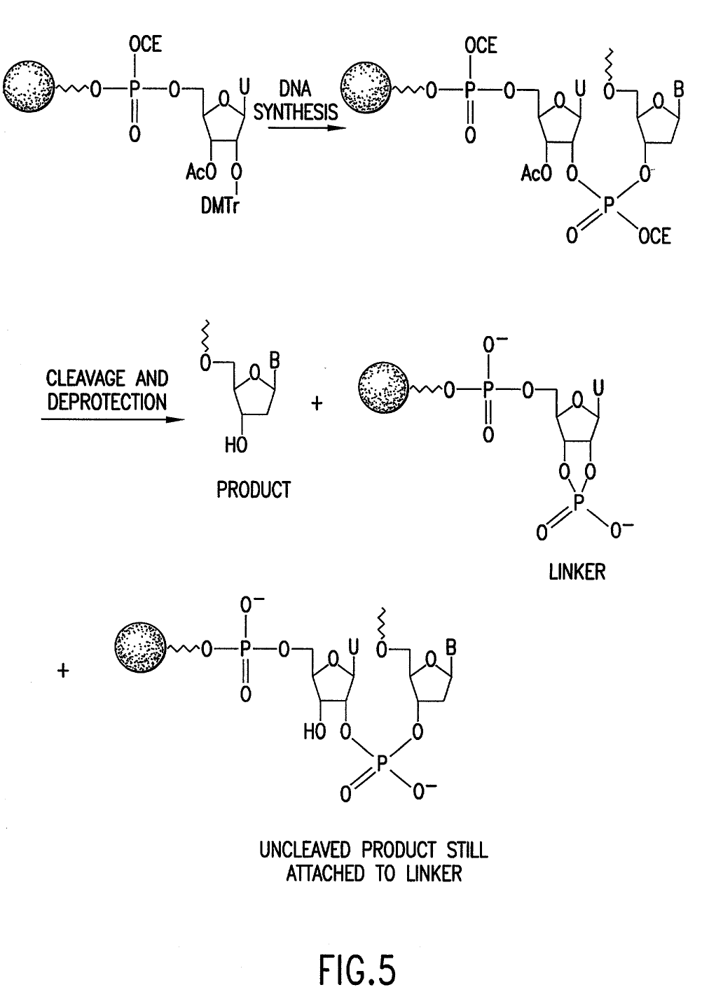

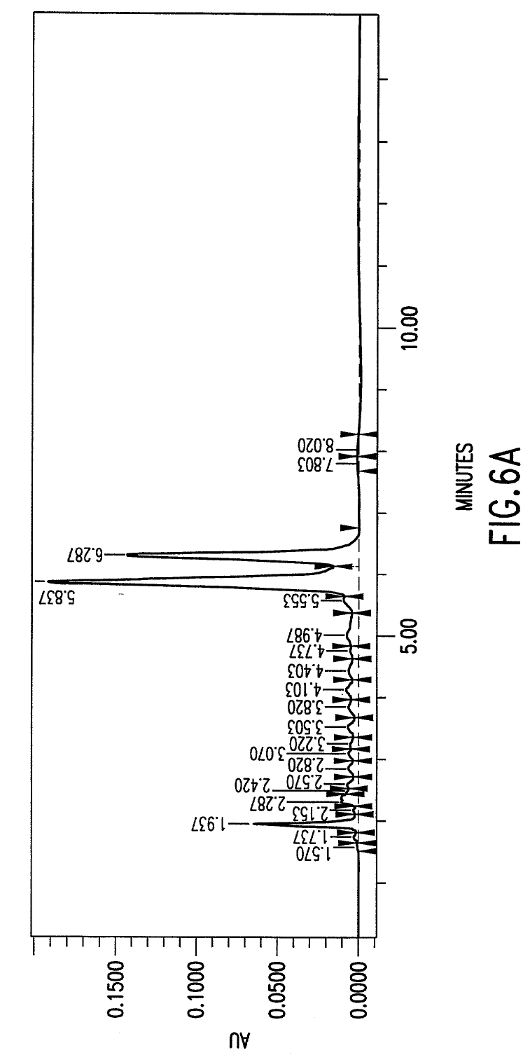

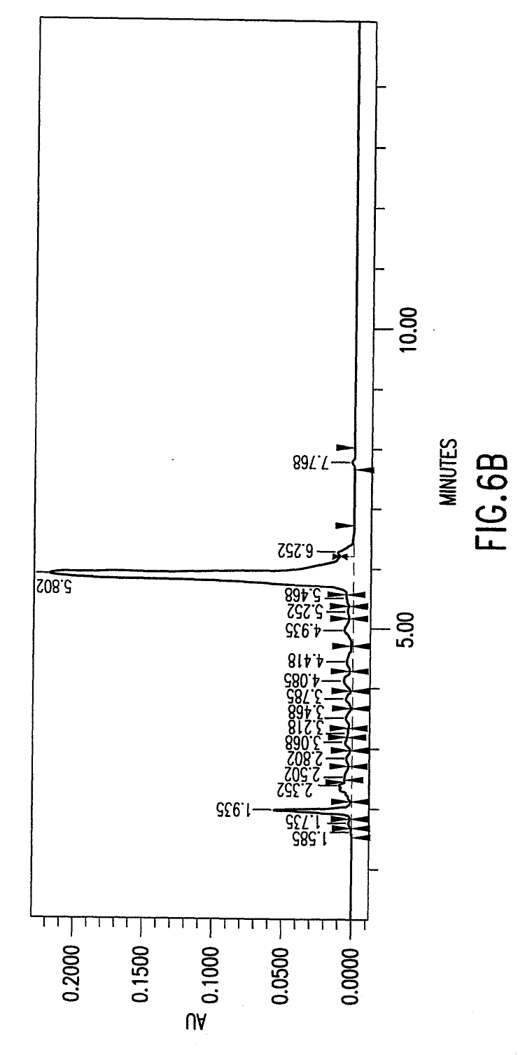

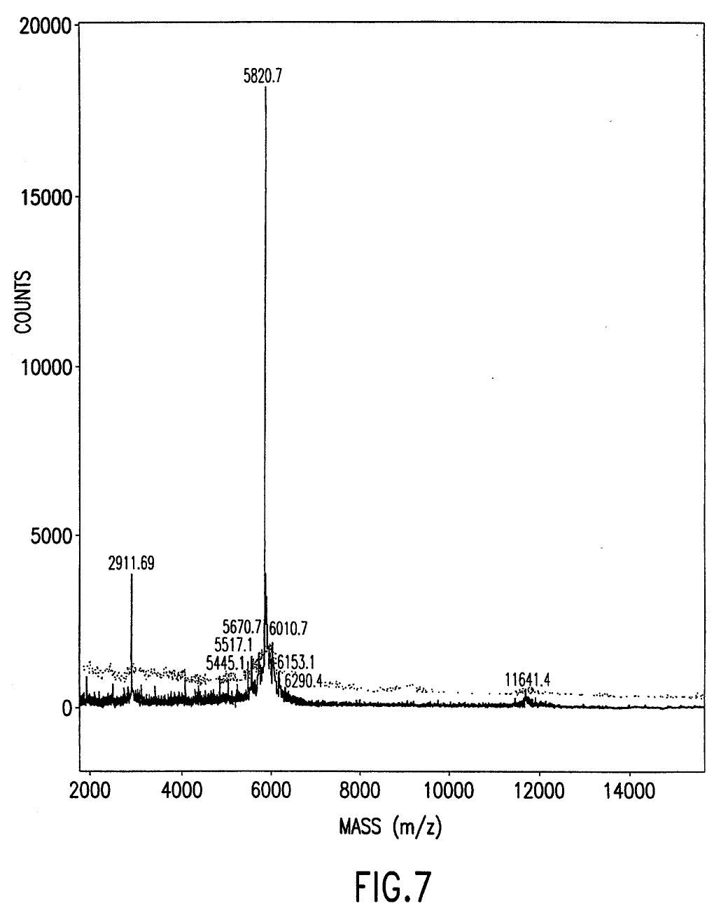

It has been discovered that gaseous ammonium hydroxide greatly accelerates the rate of cleavage of oligonucleotides from universal linkers. FIGS. 5A and 5B depict HPLC comparisons of gas phase cleavage and deprotection at 95° C., 80 psi, 60 min of 20-mer (FIG. 6A) with concentrated ammonium hydroxide at 95° C. and 75 min (FIG. 6B). This brings the possibility of using a universal linker in a high throughput environment within grasp.

The following sequences were synthesized on a high throughput parallel DNA synthesizer, using Universal Support Type 2 (polystyrene) from Biosearch Technologies, Inc.

| | | |
|---|---|---|
| 19 mer: | 5′ - TTC AGC AAG CGA CTA GTG T - 3′ | (SEQ ID NO: 1) |
| 59 mer: | 5′ - TTC AGC AAG CGA CTA GTG TCT TCA GCA AGC | (SEQ ID NO: 2) |
| | GAC TAG TGT CTT CAG CAA GCG ACT AGT GT - 3′ | |

After synthesis, the oligos were placed in a high pressure reactor containing an inlet vent for gas, an outlet vent, and a safety release valve. The vessel was pre-equilibrated at 95 ° C. After sealing the chamber, the gas phase reactor was filled with hydrated ammonia gas until the pressure reached 80 PSI. This pressure was maintained for 1.5 hours. The gas was then released through a vent and the oligos were removed from the chamber. The oligos were eluted from the support using water, and analyzing by ion pairing HPLC using a C₈-column. (65% A to 35% B over 13 minutes. A is 20 mM NaH₂PO₄, 5 mM tetrabutyl ammonium phosphate. Solvent B is acetonitrile). The peak at 1.9 minutes is from benzamide.

All publications, patents and patent applications mentioned in this specification are indicative of the level of skill of those in the art to which the invention pertains. All publications, patents and patent applications are herein incorporated by reference to the same extent as if each individual publication or patent application was specifically and individually indicated to be incorporated by reference in their entirety.

What is claimed is:

1. A method for cleavage of a linker from an oligonucleotide, comprising contacting a conjugate comprising an oligonucleotide, a linker and a solid support with a gaseous nucleophilic composition under conditions that result in the cleavage of the linker from the oligonucleotide.

2. The method of claim 1, wherein said linker is a universal linker.

3. The method of claim 1, wherein said linker which attaches the oligonucleotide to the solid support is not the 3′-terminal nucleotide.

4. The method of claim 1, wherein the linkage being cleaved is an ester linkage between the 3′-OH of the oligonucleotide and phosphate of the linker.

5. The method of claim 4, wherein the linker, when removed, produces a phosphorous containing heterocycle.

6. The method of claim 1, wherein said linker contains 2 vicinal heteroatoms.

7. The method of claim 1, wherein said linker comprises a vicinal diol.

8. The method of claim 1, wherein said linker comprises a vicinal amino alcohol.

9. The method of claim 1, wherein said linker comprises a vicinal thiol alcohol.

10. The method of claim 1, wherein said gaseous nucleophilic compound is ammonia vapors.

11. The method of claim 1, wherein said gaseous nucleophilic compound is hydrated ammonia vapors.

12. The method of claim 1, wherein said conditions comprise carrying out the process for about 1 minute to 240 minutes.

13. The method of claim 11, wherein said conditions comprise carrying out the process for about 60 minutes.

14. The method of claim 1, wherein said conditions comprise carrying out the process at about room temperature to about 150° C.

15. The method of claim 14, wherein said conditions comprise carrying out the process at about 95° C.

16. The method of claim 1, wherein said the ester linkage between the 3′-hydroxyl of the terminal nucleotide of the oligonucleotide and the linker is substantially cleaved.

17. The method of claim 16, wherein said cleaved oligonucleotide is recovered by washing said solid phase with water or aqueous buffer.

18. A method for cleavage of a linker from an oligonucleotide, comprising contacting ammonium hydroxide vapors with a conjugate comprising a linker, an oligonucleotide and a solid support at 95° C. and 80 psi for 120 minutes, resulting in the cleavage of the linker from the oligonucleotide.

19. The method of claim 1, wherein the oligonucleotide, linker, solid support conjugate has the formula: 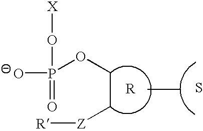
wherein X is the termini of the oligonucleotide, S is a solid support, R is an optionally substituted tetrahydrofuran, phenyl or cyclopentane ring, and R′ is a protecting group, and Z is O, S or Se.

20. The method of claim 19, wherein X is the 3′ terminal nucleotide of the oliogonucleotide.

21. Method of claim 19, wherein the protecting group is a DMTr, acyl, aryl, silyl, tripluoroacetyl, benzyl, substituted benzyl or aryl group.
